(19) United States
(12) Patent Application Publication
(10) Pub. No.: US 2008/0065065 A1
(43) Pub. Date: Mar. 13, 2008

(54) BIPOLAR COAGULATION ELECTRODE

(75) Inventor: Bert H. Sutter, Vorstetten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: SUTTER MEDIZINTECHNIK GMBH, Freiburg (DE)

(21) Appl. No.: 11/939,261

(22) Filed: Nov. 13, 2007

Related U.S. Application Data
(63) Continuation of application No. 10/839,836, filed on May 6, 2004.

(30) Foreign Application Priority Data
Mar. 19, 2004 (DE) 202004004306.8

(51) Int. Cl. A61B 18/14

(52) U.S. Cl. 606/50

(57) ABSTRACT

A bipolar coagulation electrode has electrode poles arranged at a distance from each other and one behind the other in a region of the distal electrode end in the longitudinal profile of the coagulation electrode. At least one of the two electrode poles is built to be electrically conductive and to deliver power over only a portion of its circumference and the remaining portion of its circumference is electrically insulated.

### CROSS REFERENCE TO RELATED APPLICATION

This application is a continuation of U.S. patent application Ser. No. 10/839,836, filed May 6, 2004 which is incorporated by reference as if fully set forth.

### FIELD OF INVENTION

The invention deals with bipolar coagulation electrodes.

### BACKGROUND

The invention relates to a bipolar coagulation electrode with two electrode poles arranged at a distance from each other and one behind the other in the region of the distal electrode end in the longitudinal profile of the coagulation electrode.

Such coagulation electrodes are already known and are used during surgical operations to apply bipolar, high-frequency current in order to coagulate biological tissue or for ablation of biological tissue. Coagulation electrodes are also occasionally used within a conductive medium, such as a saline solution. The known coagulation electrodes feature electrode poles at their distal end, which are arranged in the circumferential direction, i.e., which deliver current flowing in the circumferential direction, which are electrically conductive, and which are formed, e.g., as two electrically insulated coaxial elements or as two quarters of a ball at the tip of the coagulation electrode.

Through a circumferential application of a high-frequency current, an unnecessarily large amount of surrounding tissue can be damaged, especially during work in tight spaces (e.g., nose, vertebrae, etc.). This can be prevented or avoided in a known way by a bent or angulated electrode tip. Such structures, however, are complicated and expensive.

Therefore, there is the problem of creating a coagulation electrode of the type defined in the introduction, which can be easily handled and with which high-frequency current can be applied over the electrode poles without too much damage to surrounding tissue, and without requiring a complicated, bent or angulated electrode tip.

### SUMMARY

To solve this problem, in accordance with the invention at least one of the two electrode poles is configured so that it is electrically conductive and able to deliver current over only a portion of its circumference and the remaining portion of its circumference is electrically insulated. Therefore, the high-frequency current can be delivered only to one side even with a coagulation electrode without special curvature or bending or angulation of its distal end, so that the tissue on the insulated side of the coagulation electrode facing away from the electrode pole is not damaged and simultaneously the treatment area can be reached better and more selectively by the current. Here, the coagulation electrode according to the invention can be handled just as easily as known unbent coagulation electrodes with circumferential electrode poles. In addition, the power loss in a conductive medium is less for the side limited application, because the energy is transmitted only at this point or applied to the tissue where it is necessary.

In one advantageous embodiment, the surface of the first electrode pole arranged at the distal end of the coagulation electrode is bare in the circumferential direction and electrically conductive, the second electrode pole at a distance from the distal end is electrically insulated over a portion of its circumference, and the surface of the first electrode pole, which delivers the power and is electrically conductive, is greater than the electrically conductive surface of the second electrode pole. In particular, for use within a conductive medium, a current density, which is different and also distributed non-uniformly over the electrode surface, is achieved at the electrode poles, with the current density at the smaller second electrode pole at a distance from the distal end being greater per surface unit than the current density at the larger first electrode pole at the distal end of the coagulation electrode. Due to the higher current density, the energy applied to the tissue at the second electrode pole can also be correspondingly high. The distance between both electrode poles insulates both electrode poles from each other, wherein the electrical properties of the coagulation electrode, especially the field distribution around the electrode poles, can be changed selectively also by varying this distance.

In an alternative, preferred embodiment, the surface of the first electrode pole and the surface of the second electrode pole spaced at a distance from the first electrode pole and from the distal end of the coagulation electrode are configured to be electrically conductive and to deliver power over only a portion of their circumference, the electrically conductive surfaces of the electrode poles delivering power are approximately flush with each other in the longitudinal direction of the coagulation electrode, and the electrically conductive surfaces of the two electrode poles delivering power are approximately the same size. Therefore, the coagulation electrode is active on only one side of its entire longitudinal profile and thus can be particularly gentle on the tissue not to be treated. The current density is also approximately the same size at both electrode poles due to their approximately equal size, which enables uniform application of energy with both electrode poles.

Therefore it is advantageous when the first and/or the second electrode pole is built to be electrically conductive and to deliver power over approximately one-fourth up to three-fourths, especially over approximately a half, of its circumference. In this way, on one hand the energy can be sufficiently concentrated in the areas to be treated and on the other hand the surrounding tissue is protected by the insulated circumferential areas of the coagulation electrode.

For use in surgery, it is advantageous and especially necessary that the electrically conductive surfaces of the electrode poles delivering power be formed of bio-compatible metal. Such metals, e.g., stainless steel, titanium, gold, silver, a noble metal alloy, or tungsten alloys, chromium alloys, or nickel alloys, or similar hard-metal alloys, are neither toxic nor do they promote coagulation and also do not trigger allergies or other immunological reactions. In addition, they are not corroded by sterilization chemicals.

It is advantageous when the surfaces of the electrode poles are cylindrical and/or curved like a sphere at least in certain regions. Through such a uniform curving, a smooth surface is produced, which makes the coagulation electrode easy to move, especially within the operating site, without damaging tissue. In addition, such outer curvatures can create a large surface area of the electrode poles and if necessary the coagulation electrodes can be easily pressed into the tissue with the curvature, so that the entire electrode pole area can be used to deliver energy.

For simple handling, it is especially advantageous if the coagulation electrode body holding the electrode poles is formed essentially of flexible material. In this way, the coagulation electrode according to the invention can be easily introduced into the operating site and in general can be moved easily, so that tissue areas that are otherwise hard to reach can also be treated. Advantageously, the electrical connection lines, which are guided in the interior of the coagulation electrode to the electrode poles, can also be flexible. In particular, for use in the working channel of an operating instrument, e.g., an endoscope, a flexible, pliant configuration of the coagulation electrode according to the invention is advantageous.

### BRIEF DESCRIPTION OF THE DRAWINGS

In the following, two embodiments of the invention are described in more detail with reference to the drawings, which are partial schematic representations. In the drawings:

FIG. 1 is a perspective view of a bipolar coagulation electrode according to the invention with a circumferential electrode pole at the distal end and an electrode pole, which is located at a distance from the distal end and which delivers power and is electrically conductive over only a portion of the circumference,

FIG. 2 is a side view according to FIG. 1,

FIG. 3 is a perspective view of the coagulation electrode with two electrode poles, which are flush with each other and which deliver power and are electrically conductive over only a portion of the circumference, and

FIG. 4 is a side view according to FIG. 3.

### DETAILED DESCRIPTION OF THE PREFERRED EMBODIMENTS

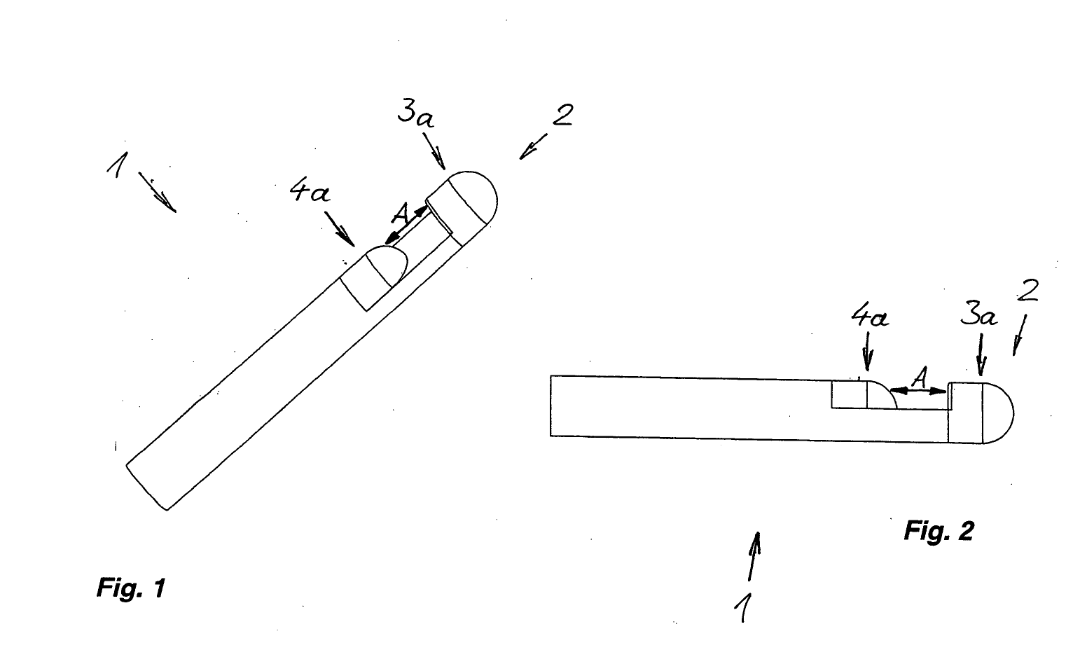

A bipolar coagulation electrode designated overall with 1 includes two electrode poles arranged at a distance from each other and one behind the other in the region of the distal electrode end 2 in the longitudinal profile of the coagulation electrode 1. In the embodiments shown in FIGS. 1-4, the coagulation electrode 1 is in the unbent position, i.e., in its original, not-in-use position configured essentially in a straight line, wherein the two electrode poles are arranged in a line one behind the other in the direction of the longitudinal axis of the coagulation electrode 1.

In the embodiment shown in FIGS. 1 and 2, the surface of the first electrode pole 3a arranged at the distal end 2 of the coagulation electrode 1 is formed bare and electrically conductive in the circumferential direction and the second electrode pole 4a at a distance from the distal end 2 is electrically insulated over a portion of its circumference. The first electrode pole 3a is here shaped partially cylindrically as a section of the coagulation electrode body and partially as a hemispherical distal end of the coagulation electrode 1.

The second electrode pole 4a is curved over approximately half of the circumference of the coagulation electrode 1, likewise partially cylindrical and partially spherical, but in comparison with the first, circumferential electrode pole 3a, it features overall a smaller surface area, in this example approximately half as large. Therefore, the current density on the second electrode pole 4a is greater than on the first electrode pole 3a, because the electromagnetic field present between the electrode poles 3a, 4a is not formed equally on the electrode poles 3a, 4a and is concentrated on the smaller surface area of the electrode pole 4a. The current density is also distributed differently over the surface of the first electrode pole 3a. Thus, a very low current density is created on the opposite side facing away from the second electrode pole 4a, a low current density at the side directly facing the second electrode pole 4a, and a medium-size current density at the distal tip of the coagulation electrode 1.

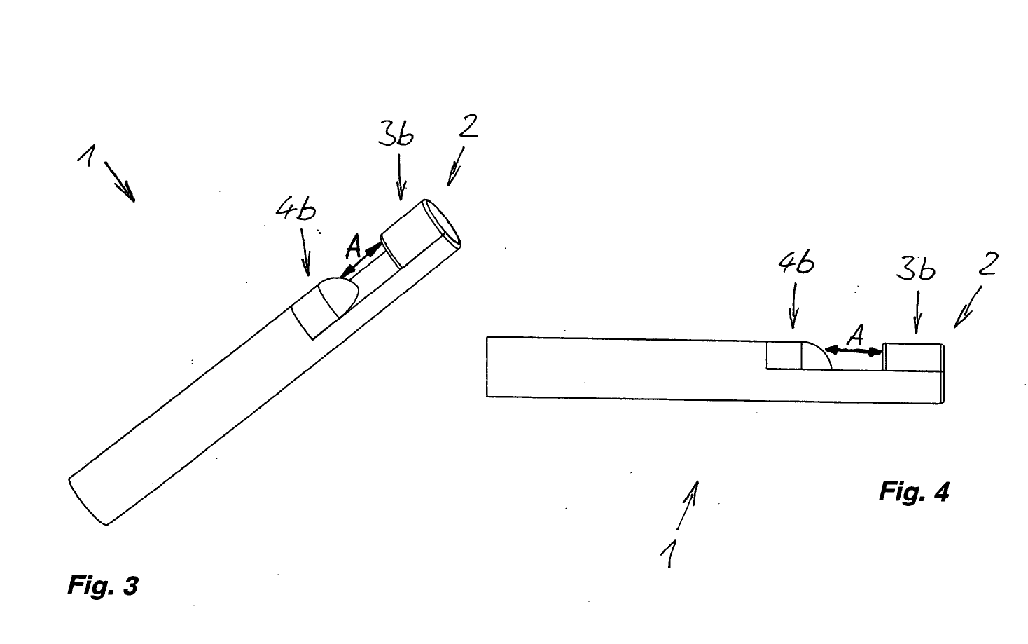

In FIGS. 3 and 4, a preferred embodiment can be seen with two electrode poles 3b, 4b with electrically conductive surfaces delivering approximately the same current magnitude. The two electrode poles 3b, 4b are both built to deliver power and to be electrically conductive over only approximately half of the circumference of the coagulation electrode 1 and to be electrically insulated over the remaining portion or the other half of the circumference. Both electrode poles 3b, 4b are flush with each other with their electrically conductive surfaces in the longitudinal direction of the coagulation electrode 1.

The surface of the first electrode pole 3b is formed as an approximately half-sided, cylindrically curved section of the coagulation electrode body. In contrast, the second electrode pole 4b has a surface, which is curved cylindrically in sections in the longitudinal direction on a circumferential region of the coagulation electrode 1 and which is curved spherically in its end region.

Through the approximately same sized surfaces of the two electrode poles 3b, 4b, the current density on the surface of both electrode poles 3b, 4b is also approximately equal, because the electromagnetic field is established approximately uniformly between both electrode poles 3b, 4b.

The distance A between the first electrode pole 3a, 3b and the second electrode pole 4a, 4b insulates both electrode poles 3a, 3b, 4a, 4b from each other, wherein the size of the distance A also determines the profile of the electromagnetic field and thus the electromagnetic properties of the coagulation electrode 1 according to the invention.

1. A bipolar coagulation electrode having a distal electrode end comprising:
a first electrode pole having a partially cylindrical shape and being electrically conductive about its circumference; and
a second electrode pole arranged at a fixed distance from the first electrode pole one behind the other in a region of the distal electrode end in a longitudinal profile of the coagulation electrode, the second electrode pole having a partially spherical shape, which is electrically conductive, and curved over approximately half of electrode's circumference and a remaining portion of the circumference is electrically insulated.

2. The bipolar coagulation electrode of claim 1, wherein the first electrode pole has a surface area that is twice as large as that of the second electrode pole.

3. The bipolar coagulation electrode of claim 1, wherein at least one of the first and second electrode poles are electrically conductive over one fourth to three fourths of its circumference.

4. The bipolar coagulation electrode of claim 1, wherein the first electrode pole delivers power and has a greater surface area than the second electrode pole.

5. A distal bipolar coagulation electrode comprising first and second electrode poles axially spaced at a fixed distance from one another, the electrode poles being electrically conductive and are arranged to deliver power only over a portion of their exterior surfaces, the surfaces that deliver power are generally identical in size and substantially flush with one another in the longitudinal direction of the coagulation electrode.

6. The coagulation electrode of claim 5, wherein the electrode has a generally cylindrical shape.

7. The coagulation electrode of claim 6, wherein the electrode poles deliver power to a partial portion of the circumference of the electrode.

8. The coagulation electrode of claim 5, wherein the first and second electrode poles are spaced at a fixed distance sufficient to electrically insulate them from one another.

9. A bipolar coagulation electrode having a generally cylindrical body and a distal electrode end, the distal electrode comprising:
a first electrode pole having a partially cylindrical shape and being electrically conductive about its circumference; and
a second electrode pole arranged at a fixed distance from the first electrode pole one behind the other in a region of the distal electrode end in a longitudinal profile of the coagulation electrode, the second electrode pole having a partially spherical shape, which is electrically conductive, and curved over approximately half of electrode's circumference and a remaining portion of the circumference is electrically insulated, the electrode poles are spaced at a fixed distance sufficient to electrically insulate them from one another and deliver power to a partial portion of the circumference of the electrode.

10. The bipolar coagulation electrode of claim 9, wherein at least one of the first and second electrode poles are electrically conductive over one fourth to three fourths of its circumference.

11. The bipolar coagulation electrode of claim 9, wherein at least one of the first and second electrode poles are electrically conductive over one half of its circumference.

12. The bipolar coagulation electrode of claim 9, wherein the first electrode pole is curved like a sphere in an end portion thereof.

13. The bipolar coagulation electrode of claim 12, wherein the entire first electrode pole delivers energy.

14. The bipolar coagulation electrode of claim 13, wherein the first electrode pole has a greater electrically conductive surface area than that of the second electrode pole.
